(19) United States
(12) Patent Application Publication
(10) Pub. No.: US 2026/0151273 A1
(43) Pub. Date: Jun. 4, 2026

(54) ARAMID REINFORCED INSTANT FLEXIBLE CAST STRIPS

(71) Applicant: MATSCITECHNO LICENSING COMPANY, KENNETT SQUARE, PA (US)

(72) Inventor: Robert A. Vito, Kennett Square, PA (US)

(21) Appl. No.: 18/966,538

(22) Filed: Dec. 3, 2024

(51) Int. Cl. A61F 13/04; A61F 13/00; A61F 13/02; A61L 15/12; A61L 15/14; C09J 7/21

(52) U.S. Cl. CPC A61F 13/04; A61F 13/00059; A61F 13/00063; A61F 13/0259; A61F 13/0273; A61F 13/0283; A61L 15/125; A61L 15/14; C09J 7/21; A61F 2013/00604; A61F 2013/00889; C09J 2301/122; C09J 2423/046

(57) ABSTRACT

A reinforced material is described. The reinforced material includes a middle layer formed of a plurality of fibers comprising a first and second side, a top layer disposed on the first side comprising an elastomeric material, and a bottom layer disposed on the second side comprising an adhesive material, wherein the reinforced material is configured to have a maximum stretch of up to 10%. In some embodiments, the reinforced material may be formed into strips or tape. A method of manufacturing a reinforced material is also described.

### BACKGROUND OF THE INVENTION

Reinforced materials have long been employed in the fields of sports, medicine, and rehabilitation to provide support, stability, and protection for injured or vulnerable body parts. “Casting” is the typical traditional treatment for injured limbs. It is typically initiated approximately one week after the fracture, once initial swelling has subsided. Casting utilizes fiberglass material for casting, although plaster may be used in certain cases.

Alternatively, pre-formed splint cast braces are an alternative method that uses a thermoplastic that when heated converts from a rigid state to a very pliable and conformable material (plaster-like). After sculpting to a desired position and shape, it reverts to a high strength immobilizer. However, these braces are pre-formed for specific body parts such as a wrist or ankle. Their bulky size and shape make them impractical to carry for a soldier or athlete to carry on their person while in the field.

Athletic tapes, splints, and braces often incorporate fibers, polymers, or composite layers to enhance their durability and effectiveness. These materials are commonly used to stabilize joints, prevent excessive movement, and assist in recovery during athletic activities or physical exertion. They are also critical in emergency settings, where quick, effective immobilization can make the difference between further injury and successful triage.

However, current reinforced materials face significant limitations. Many are either too rigid, severely restricting mobility, or excessively elastic, stretching beyond 100% of their original length, which compromises their ability to immobilize and support injuries effectively. Overly flexible materials fail to maintain the necessary stability during prolonged physical activity, while overly rigid materials can cause discomfort and hinder natural movements. This lack of control in stretch and rigidity of reinforced materials has created the need for materials that can.

Thus, there is a need in the art for reinforced materials for sports, medicine and rehabilitation, in order to provide precise, predictable support to a subject while remaining easy to use and adaptable to diverse environments. It must be a precise customized fit that is easily and quickly achieved in length and circumference, while securing anatomical conformity for repair preservation. The present disclosure satisfies that need.

### SUMMARY OF THE INVENTION

A reinforced material including a middle layer formed of a plurality of fibers comprising a first and second side, a top layer disposed on the first side comprising an elastomeric material, and a bottom layer disposed on the second side comprising an adhesive material, wherein the reinforced material is configured to have a maximum stretch of up to 10%. In some embodiments, the plurality of fibers comprise any of aramid fibers, high tensile strength fibers, or a versatile, low-weight, high-strength high modulus polyethylene fibers. In some embodiments, the plurality of fibers are pre-stretched. In some embodiments, the pre-stretch of the plurality of fibers ranges between 50% and 99% of the maximum stretch of the fibers. In some embodiments, the maximum stretch is a 2-way stretch or a 4-way stretch.

In some embodiments, the reinforced material includes a removable or peelable layer disposed on the top or bottom layer. In some embodiments, the removable or peelable layer comprises a protective release liner removably attached to the bottom layer to preserve the adhesive material prior to use. In some embodiments, the plurality of fibers are arranged in a unidirectional configuration, a multi-directional configuration, or one or more woven patterns to maximize tensile strength along one or more axis. In some embodiments, the one or more woven patterns comprises warp and weft fibers, each having a thread count or number of fibers per unit area, wherein the thread count or number of fibers per unit area of the weft fiber is less than the thread count or number of fibers per unit area of the warp fibers.

In some embodiments, the reinforced material includes elastic fibers woven into the middle layer. In some embodiments, the reinforced material is configured to be hypoallergenic and suitable for prolonged contact with human skin. In some embodiments, the elastomeric coating is configured to provide a textured or high-friction surface to improve grip and handling during application. In some embodiments, the reinforced material is fabricated in the form of a roll, a roll of strips, a patch, or a strip for specific applications. In some embodiments, the strip is at least partially formed in an elongate shape, a winged-shape, an hourglass shape, or an irregular shape.

In some embodiments, the top layer is treated or configured to be water-resistant or moisture-wicking. In some embodiments, the reinforced material is perforated to allow for ventilation during use, or has one or more cutout regions configured as reliefs. In some embodiments, the adhesive material comprises an antimicrobial agent. In some embodiments, the elastomeric coating is lettered, colored or patterned to indicate specific application zones, instructions, cutting zones, or to provide aesthetic customization.

In some embodiments, the reinforced material is configured to be cut using standard scissors or cutting tools without fraying or delaminating. In some embodiments, the reinforced material is configured to be waterproof, moisture-proof, and sweat-proof. A roll of tape formed from pre-cut strips of reinforced material, the reinforced material including a middle layer formed of a plurality of aramid fibers comprising a first and second side, a top layer disposed on the first side comprising an elastomeric coating, and a bottom layer disposed on the second side comprising an adhesive coating.

A splinting device for immobilizing an appendage including a reinforced material configured to conform to the shape of an appendage or joint wherein the material is applied to the appendage or joint to restrict movement while allowing limited flexibility through a maximum stretch of 10%, and wherein the reinforced material is pre-shaped or cut to a specific size for splinting fingers, wrists, ankles, arms, or legs.

A method of fabricating a reinforced material including the steps of pre-stretching a plurality of high-tensile strength fibers into a middle layer having a first side and a second side, applying an elastomeric layer, material or coating to the first side, and applying an adhesive layer, material or coating to the second side, wherein the reinforced material is configured to have a maximum stretch of up to 10%. In some embodiments, the pre-stretch of the fibers ranges between 50% and 95% of the maximum stretch of the fibers.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIG. 1A depicts an exemplary reinforced material according to aspects of the present invention.

FIGS. 1B-1E show the reinforced material of the present invention formed into a roll of tape (e.g., athletic tape). FIG. 1B is an image showing a bottom view of the tape. FIG. 1C is an image showing a top view of the tape. FIG. 1D is an image showing a side view of the tape. FIG. 1E is an image showing an enlarged side view of the tape.

FIG. 2 shows the reinforced material of the present invention formed into Aramid reinforced instant flexible cast strips applied around an ankle to support the ankle without excessively hindering movement.

FIG. 3 shows the reinforced material of the present invention formed into Aramid reinforced instant flexible cast the strips applied around a wrist in layers to immobilize the wrist.

FIG. 4 shows a splint or cast formed by layering the reinforced material that retains its structure and form when removed from the subject, and exhibits or retains flexibility and rigidity.

FIGS. 5A & 5B show the ‘casting’ method which is the traditional treatment for injured limbs. It is typically initiated approximately one week after the fracture, once initial swelling has subsided. Casting utilizes fiberglass material for casting, although plaster may be used in certain cases.

FIG. 6 shows a pre-formed splint cast brace using a thermoplastic that when heated converts from a rigid state to a very pliable and conformable material (plaster-like). After sculpting to a desired position and shape, it reverts to a high strength immobilizer.

FIG. 7 shows a comparison of the typical immobilizer methods that includes a splint, plaster cast, and fiberglass cast.

### DETAILED DESCRIPTION

It is to be understood that the figures and descriptions of the present invention have been simplified to illustrate elements that are relevant for a clear understanding of the present invention, while eliminating, for the purpose of clarity, many other elements found in related systems and methods. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of +20%, ±10%, ±5%, ±1%, and ±0.1% from the specified value, as such variations are appropriate.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, 6 and any whole and partial increments therebetween. This applies regardless of the breadth of the range.

The terms “proximal,” “distal,” “anterior,” “posterior,” “medial,” “lateral,” “superior,” and “inferior” are defined by their standard usage indicating a directional term of reference. For example, “proximal” refers to an upper location from a point of reference, while “distal” refers to a lower location from a point of reference. In another example, “anterior” refers to the front of a body or structure, while “posterior” refers to the rear of a body or structure. In another example, “medial” refers to the direction towards the midline of a body or structure, and “lateral” refers to the direction away from the midline of a body or structure. In some examples, “lateral” or “laterally” may refer to any sideways direction. In another example, “superior” refers to the top of a body or structure, while “inferior” refers to the bottom of a body or structure. It should be understood, however, that the directional term of reference may be interpreted within the context of a specific body or structure, such that a directional term referring to a location in the context of the reference body or structure may remain consistent as the orientation of the body or structure changes.

The terms “patient,” “subject,” “individual,” and the like are used interchangeably herein, and refer to any animal amenable to the systems, devices, and methods described herein. The patient, subject or individual may be a mammal, and in some instances, a human.

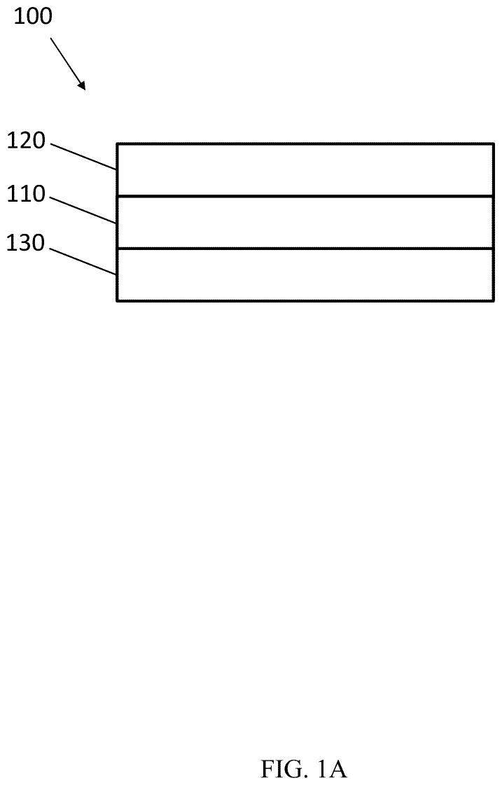

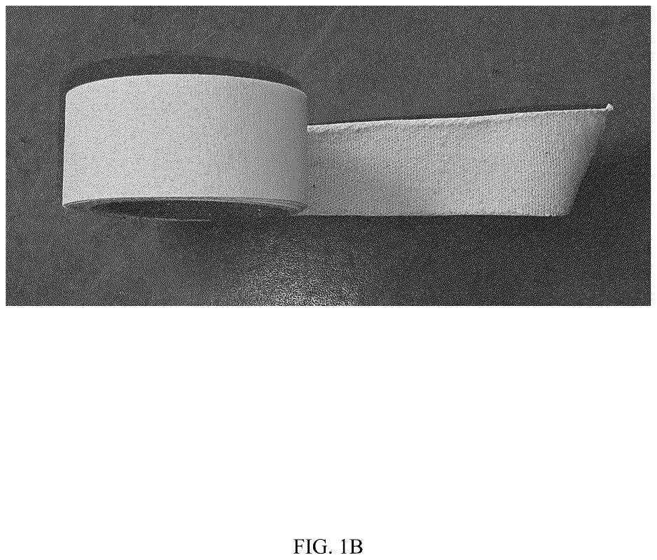

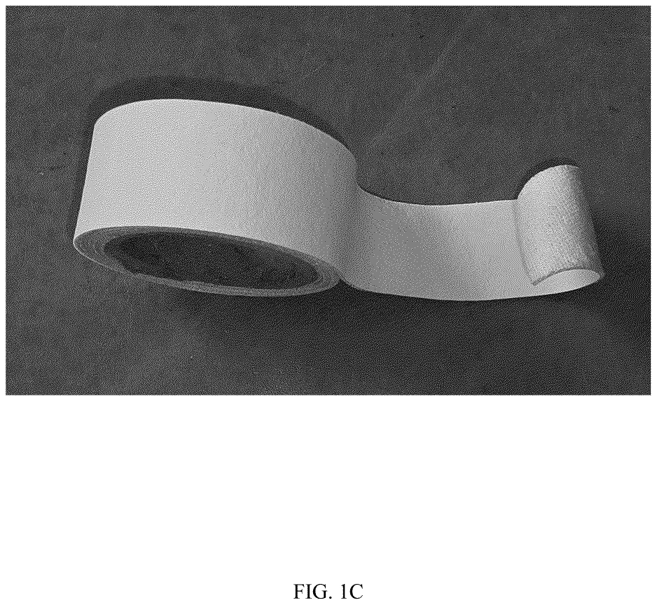

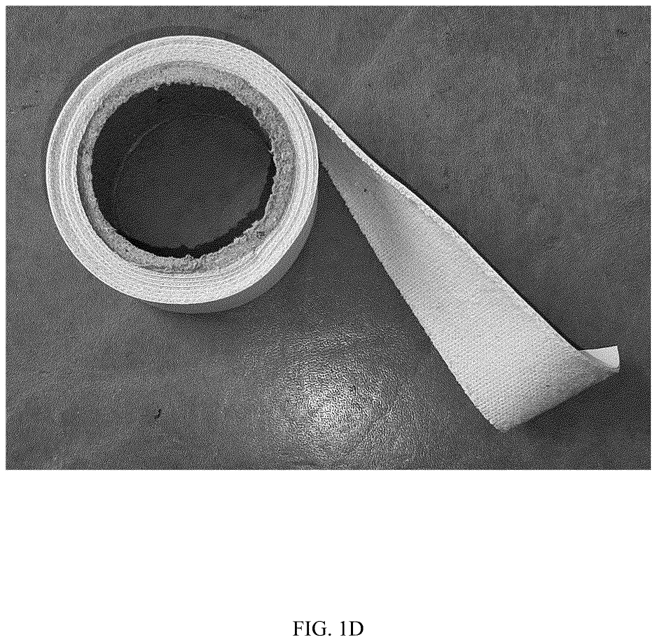

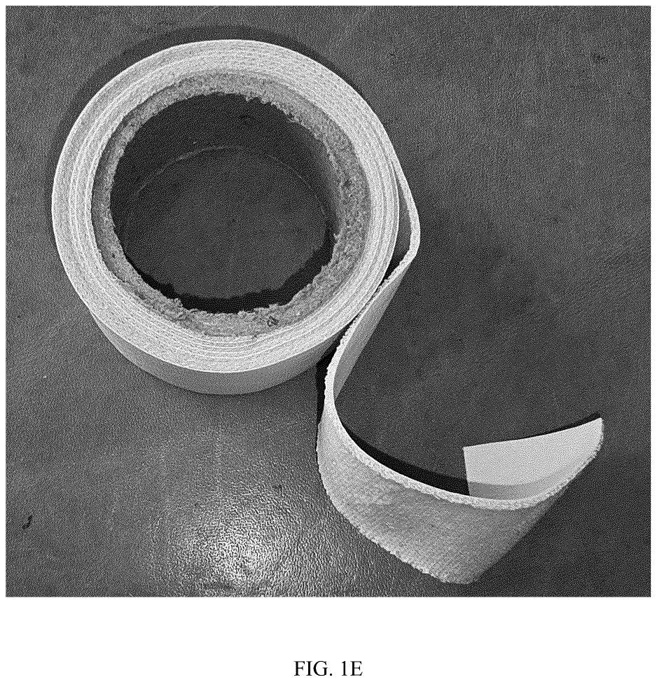

Aspects of the present disclosure relate to reinforced material (e.g., a multilayered aramid-reinforced material) designed to provide superior control and stability with a pre-set maximum stretch or elongation. Referring now to FIG. 1A, shown is a diagram depicting an exemplary reinforced material 100 according to aspects of the present invention. In some embodiments, reinforced material 100 comprises a middle layer 110 with a top layer 120 positioned or disposed on a first side of the middle layer 110, and a bottom layer 130 positioned or disposed on a second side of the middle layer 110. In some embodiments, the reinforced material 100 comprises a cloth, fabric or textile forming the middle layer 110. In some embodiments, the reinforced material 100 comprises a middle layer 110 of fibers for high tensile strength (e.g., aramid fibers), a top layer 120 comprising an elastomeric layer, material, and/or coating, and a bottom layer 130 comprising an adhesive material, layer, and/or coating that allows secure attachment to surfaces such as skin or clothing. The disclosed reinforced material 100 comprises a unique composite structure that offers a semi-flexible yet firm solution for supporting or immobilizing joints or injuries, ensuring that movement is minimized while maintaining enough flexibility for natural activity. The elastomeric layer or coating provides resilience and durability for the material, while the aramid fiber core ensures consistent reinforcement, with an adhesive layer that facilitates easy application and secure placement.

In some embodiments, an exemplary middle layer 110 comprises a cloth or weave formed of a plurality of fibers (e.g., woven aramid fibers). In some embodiments, the fibers comprise any of aramid fibers, polyethylene fibers, high tensile strength fibers, or a versatile, low-weight, high-strength high modulus polyethylene fibers, or the like. In some embodiments, the middle layer 110 is a weave or random distribution of fibers (e.g., aramid fibers). In some embodiments, the middle layer 110 comprises one or more layers, weaves and/or random distributions of fibers (e.g., aramid fibers), or one or more pluralities of fibers. In some embodiments, the one or more plurality of fibers are formed in a weave, pattern or in one or more bundles. In some embodiments, the middle layer 110 comprises a percentage of floating fibers, that is, it may be preferable that at least some of the plurality of fibers are able to move relative to the remaining fibers of the cloth or layer. In some embodiments, an elastomeric layer (e.g., top layer 120) at least partially covers the middle layer 110, and/or at least partially or fully intrudes into or saturates the middle layer 110. In some embodiments, first and second elastomeric layers at least partially surround or cover the middle layer. In some embodiments, the elastomeric or top layer 120 is patterned on the middle layer 110, and/or exposes portions of the middle layer 110. In some embodiments, an adhesive layer or coating is applied to at least one of the elastomeric layers and/or at least partially to an exposed portion of the middle layer 110, or the entire middle layer 110. In some embodiments, the fibers (e.g., aramid fibers) are coated in an antimicrobial coating, anti-static coating, and/or an anti-friction coating. In some embodiments, any of the bottom layer 130, middle layer 110 or top layer 120 comprise more than one material, layer or coating. For example, the top layer 120 may be formed of one or more elastomeric materials with a coating applied as discussed herein, or the bottom layer 130 may be formed of a material layer or foam layer coated in an adhesive coating or layered with an adhesive layer. In some embodiments, the bottom layer 130, middle layer 110 or top layer 120 of the reinforced material 100 comprises a padding layer, a cushion, or a foam layer. In some embodiments, the reinforced material has shape memory, particularly when layered.

Reinforced material 100 may be formed of or include any known elastomeric materials.

Generally, the top layer 120 comprises materials that are less reactive and cannot be cured with sulfur vulcanization, and are configured to withstand damage from sunlight, oxidizing materials, ozone, heat, and many oxygenated solvents. In some embodiments, top layer 120 comprises one or more elastomeric materials such as synthetic elastomer, butyl rubber, Isobutylene-isoprene (IIR), saturated elastomers, or the like. Reinforced material 100 may be formed of or include any known adhesive materials such as pressure-sensitive adhesive (PSA). Generally, any soft polymer or polymer blend that adheres to surfaces through physical forces like van der Waals forces when pressure is applied may be used. Importantly, the adhesive sticks without needing to dry or cure like traditional glue. In some embodiments, bottom layer 130 comprises one or more adhesive materials such as a pressure-sensitive adhesive (PSA), natural or synthetic rubber adhesives, silicon adhesives, acrylic adhesives, or the like.

In some embodiments, the reinforced material is formed into aramid reinforced strips that have a customized range of elongation of as little as 1% to a maximum stretch of up to 10%.

Unlike traditional athletic tape that will continue to stretch up to or greater than 100%, the disclosed reinforced material can be formed into a strip will only elongate to an additional 10%. This unprecedented control enables the trainer or applicator to immobilize the joint or injured area and hold in place throughout hours of play in a game, a hiker in the woods, a rock climber in the wilderness, or a soldier walking off a battlefield.

Generally, the disclosed reinforced material 100 forms a textile, a fabric, strips, or a tape that has a maximum stretch or elongation. For example, in some embodiments, the material will only stretch or elongate 10% per unit of material. This elongation can be customized to be greater or less by the selection of the fibers (e.g., type of fibers, density and size of fibers). For example, in some embodiments, the fibers are chosen and configured to have a 7% elongation.

Or, in some embodiments, the fibers have a minimal 1% to 3% elongation. In one example, a 20 cm strip or length of tape has a maximum stretch of about 2 cm. In a non-limiting example, the disclosed reinforced material, or pre-cut strips thereof, may be formed into a roll of tape (e.g., athletic tape). It should be appreciated that the weave, density, thread count, number of vertical threads (warp), number of horizontal threads (weft), fill, thickness of layers, width of layer, thickness of reinforcing fibers or strands (e.g., Kevlar®) may all influence and/or modulate the maximum stretch or elongation of the reinforced material. In some embodiments the maximum stretch ranges between about 1% and about 25%, or is about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, or about 25%. Although exemplary maximum stretches are discussed herein, the disclosed reinforced material may be configured for any stretch or elongation, such as up to a maximum stretch of 50%, 75%, or 100%. In some embodiments, different portions of the reinforced material 100 may exhibit different maximum stretch or elongation, as discussed herein.

In some embodiments, the reinforced material 100 is formed into pre-cut strips that may be formed into one or more shapes, depending on the application. The reinforced material 100 may be formed into any known shape or configuration for kinesiology tape, athletic tape, or strips known by one of ordinary level of skill int the art. For example, an X-shaped strip for being applied over a knee-cap, or winged or hourglass shape for applying over the shoulder, or around the ankle or heel. Depending on the shape of the reinforced material 100, there may be one or more axis that the material immobilizes or supports with a maximum stretch or elongation. For example, an x-shaped pre-cut strip may provide reinforcement with a maximum stretch along 2 intersecting axes correlating to each portion of the “x”. Further, in this configuration, the reinforced material comprises one or more patterns or general directions for the plurality of fibers. It should be appreciated that any number of layers of fibers, or layers of plurality of fibers, oriented in any direction may be layered upon one another to form the middle layer and/or a shape of the disclosed reinforced material 100. Further, each pattern or general direction for the fibers or plurality of fibers in the middle layer 110 may have a pre-stretch, or may be held taught or elongated while being formed. In one example, to provide one or more axes of support to a wing or hourglass shaped strip, a first plurality of fibers extends along the length of the strip, and one or more additional plurality of fibers are layered on the first plurality in directions correlating to the shape of the strip, or correlating to portions on a subject that the strip intends to immobilize or support. Each plurality of fibers may have the same pre-stretch, or maximum stretch, or may have a different pre-stretch, or maximum stretch. For example, the hourglass shape may have a maximum stretch of up to 10% along the length of the strip, and then one or more different stretches along a different axis from the length that may range between 1% and 25%. When forming a complex shaped reinforced material 100, the various plurality of fibers may be adhered to one another, prior to applying the elastomeric layer, or adhesive layer. However, it should be appreciated that complex shapes of the reinforced material may be formed by a user or the subject, simply by layering and patterning strips over a portion of the subject to immobilize or support.

FIGS. 1B-1E show the reinforced material of the present invention formed into a roll of tape (e.g., athletic tape). The reinforced material can be manufactured and utilized in various forms to suit different applications. It can be fabricated as a fabric or textile for wrapping injured areas, produced in rolls of tape for versatile and customizable applications, or shaped into pre-cut strips, bandages, or patches for specific uses.

In some embodiments, pre-cut strips of reinforced material 100 are rolled into a roll of tape. The strips can be formed into any shape, such as circular, round, oval, square, rectangular, polygonal, oblong, elongate, irregular shape, x-shaped, crescent shape, hourglass shape, and any combinations thereof. In some embodiments, the strip may be formed into a base pad with extensions or fingers extending out laterally from the base of the strip. In some embodiments, the strips may comprise one or more cut out portions or reliefs, or patterns of cut out portions, holes, or reliefs. Additionally, by layering the strips or tape, a splint or cast may be formed to stabilize and/or immobilize fractures or severe sprains. The adaptability of the reinforced material extends to emergency field use, sports medicine, and everyday personal injury care, offering a practical and effective alternative to traditional athletic tapes and splints. Its compact size makes it practical and portable and instant. The disclosed reinforced material 100 is also configured to be impervious to moisture such as water and sweat which rapidly degrade the effectiveness of traditional tapes, splints and casts. High tensile strength fiber are not easily cuttable often requiring a special cutting tool, but the disclosed reinforced material 100 suspends these high tensile strength fibers in a composite that enable this invention to be cut to size with an ordinary scissor or knife.

Reinforced material 100 may comprise a composite material for middle layer 110 comprising any known fibers in any known weave, pattern or construction. For example, a middle layer 110 comprising a warp fiber and fill fiber arranged in a plain weave pattern. In some embodiments, middle layer 100 comprises a 195 denier fiber and/or 200 denier fiber with open weave pattern comprising a low construction such as a 10×10 plain weave construction or 10×15 plain weave construction. Any denier or size fiber may be used, such as between 40 denier and 600 denier.

In some embodiments, the fibers have a size or diameter ranging between about 10 nm and about 250000 nm, about 10 nm and about 100000 nm, about 10 nm and about 10000 nm, or about 10 nm and about 1000 nm, or between about 0.001 mm to about 0.2 mm, or about 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, or about 20 μm.

In some embodiments, the middle layer 110 has a thickness ranging between about 0.01 mm and 5 mm, about 0.1 mm and 3 mm, about 0.1 and 2 mm, or has a thickness of about 0.05 mm, 0.10 mm, 0.15 mm, 0.20 mm, 0.25 mm, 0.30 mm, 0.35 mm, 0.40 mm, 0.45 mm, 0.50 mm, 0.55 mm, 0.60 mm, 0.65 mm, 0.70 mm, 0.75 mm, 0.80 mm, 0.85 mm, 0.90 mm, 0.95 mm, 1.00 mm, 1.05 mm, 1.10 mm, 1.15 mm, 1.20 mm, 1.25 mm, 1.30 mm, 1.35 mm, 1.40 mm, 1.45 mm, 1.50 mm, 1.55 mm, 1.60 mm, 1.65 mm, 1.70 mm, 1.75 mm, 1.80 mm, 1.85 mm, 1.90 mm, 1.95 mm, 2.00 mm, 2.05 mm, 2.10 mm, 2.15 mm, 2.20 mm, 2.25 mm, 2.30 mm, 2.35 mm, 2.40 mm, 2.45 mm, 2.50 mm, 2.55 mm, 2.60 mm, 2.65 mm, 2.70 mm, 2.75 mm, 2.80 mm, 2.85 mm, 2.90 mm, 2.95 mm, or about 3.00 mm.

In some embodiments, the elastomeric or top layer 120 has a thickness ranging between about 0.001 mm and about 3 mm, about 0.01 mm and about 2 mm, or about 0.01 and 1 mm, or has a thickness of about 0.05 mm, 0.10 mm, 0.15 mm, 0.20 mm, 0.25 mm, 0.30 mm, 0.35 mm, 0.40 mm, 0.45 mm, 0.50 mm, 0.55 mm, 0.60 mm, 0.65 mm, 0.70 mm, 0.75 mm, 0.80 mm, 0.85 mm, 0.90 mm, 0.95 mm, 1.00 mm, 1.05 mm, 1.10 mm, 1.15 mm, 1.20 mm, 1.25 mm, 1.30 mm, 1.35 mm, 1.40 mm, 1.45 mm, 1.50 mm, 1.55 mm, 1.60 mm, 1.65 mm, 1.70 mm, 1.75 mm, 1.80 mm, 1.85 mm, 1.90 mm, 1.95 mm, 2.00 mm, 2.05 mm, 2.10 mm, 2.15 mm, 2.20 mm, 2.25 mm, 2.30 mm, 2.35 mm, 2.40 mm, 2.45 mm, 2.50 mm, 2.55 mm, 2.60 mm, 2.65 mm, 2.70 mm, 2.75 mm, 2.80 mm, 2.85 mm, 2.90 mm, 2.95 mm, 3.00 mm, 3.05 mm, 3.10 mm, 3.15 mm, 3.20 mm, 3.25 mm, 3.30 mm, 3.35 mm, 3.40 mm, 3.45 mm, 3.50 mm, 3.55 mm, 3.60 mm, 3.65 mm, 3.70 mm, 3.75 mm, 3.80 mm, 3.85 mm, 3.90 mm, 3.95 mm, 4.00 mm, 4.05 mm, 4.10 mm, 4.15 mm, 4.20 mm, 4.25 mm, 4.30 mm, 4.35 mm, 4.40 mm, 4.45 mm, 4.50 mm, 4.55 mm, 4.60 mm, 4.65 mm, 4.70 mm, 4.75 mm, 4.80 mm, 4.85 mm, 4.90 mm, 4.95 mm, or about 5.00 mm.

In some embodiments, the adhesive or bottom layer 130 layer has a thickness ranging between about 0.001 mm and about 5 mm, about 0.01 mm and about 2 mm, or about 0.1 and about 1 mm, or has a thickness of about 0.05 mm, 0.10 mm, 0.15 mm, 0.20 mm, 0.25 mm, 0.30 mm, 0.35 mm, 0.40 mm, 0.45 mm, 0.50 mm, 0.55 mm, 0.60 mm, 0.65 mm, 0.70 mm, 0.75 mm, 0.80 mm, 0.85 mm, 0.90 mm, 0.95 mm, 1.00 mm, 1.05 mm, 1.10 mm, 1.15 mm, 1.20 mm, 1.25 mm, 1.30 mm, 1.35 mm, 1.40 mm, 1.45 mm, 1.50 mm, 1.55 mm, 1.60 mm, 1.65 mm, 1.70 mm, 1.75 mm, 1.80 mm, 1.85 mm, 1.90 mm, 1.95 mm, 2.00 mm, 2.05 mm, 2.10 mm, 2.15 mm, 2.20 mm, 2.25 mm, 2.30 mm, 2.35 mm, 2.40 mm, 2.45 mm, 2.50 mm, 2.55 mm, 2.60 mm, 2.65 mm, 2.70 mm, 2.75 mm, 2.80 mm, 2.85 mm, 2.90 mm, 2.95 mm, 3.00 mm, 3.05 mm, 3.10 mm, 3.15 mm, 3.20 mm, 3.25 mm, 3.30 mm, 3.35 mm, 3.40 mm, 3.45 mm, 3.50 mm, 3.55 mm, 3.60 mm, 3.65 mm, 3.70 mm, 3.75 mm, 3.80 mm, 3.85 mm, 3.90 mm, 3.95 mm, 4.00 mm, 4.05 mm, 4.10 mm, 4.15 mm, 4.20 mm, 4.25 mm, 4.30 mm, 4.35 mm, 4.40 mm, 4.45 mm, 4.50 mm, 4.55 mm, 4.60 mm, 4.65 mm, 4.70 mm, 4.75 mm, 4.80 mm, 4.85 mm, 4.90 mm, 4.95 mm, or about 5.00 mm.

In some embodiments, the overall thickness of an exemplary reinforced material 100 is 0.0625″ or about 1.6 mm thick. In some embodiments, top layer 120 is comprised of an elastomeric or top layer 120 that is about 2 mm- 5 mm thick after the elastomer has been absorbed by and saturated the middle layer 110 (e.g., the high tensile fiber layer). In some embodiments, the bottom layer 130 is about 2 mm-5 mm thick.

In some embodiments, the strips have a width that range between about 5 mm and about 100 mm, about 10 mm and 80 mm, or greater than 100 mm, or has a width of about 5 mm, 10 mm, 15 mm, 20 mm, 25 mm, 30 mm, 35 mm, 40 mm, 45 mm, 50 mm, 55 mm, 60 mm, 65 mm, 70 mm, 75 mm, 80 mm, 85 mm, 90 mm, 95 mm, or about 100 mm. In some embodiments, the strips have a length ranging between about 25 mm and about 1000 mm, or has a length of about 25 mm, 50 mm, 75 mm, 100 mm, 125 mm, 150 mm, 175 mm, 200 mm, 225 mm, 250 mm, 275 mm, 300 mm, 325 mm, 350 mm, 375 mm, 400 mm, 425 mm, 450 mm, 475 mm, 500 mm, 525 mm, 550 mm, 575 mm, 600 mm, 625 mm, 650 mm, 675 mm, 700 mm, 725 mm, 750 mm, 775 mm, 800 mm, 825 mm, 850 mm, 875 mm, 900 mm, 925 mm, 950 mm, 975 mm, or about 1000 mm.

Aspects of the present invention relate to the fabrication or manufacture of a reinforced material. For example, in some embodiments, an exemplary method of fabricating a reinforced material comprises the steps of pre-stretching a plurality of high-tensile strength fibers into a middle layer having a first side and a second side, applying an elastomeric layer, material or coating to the first side, and applying an adhesive layer, material or coating to the second side, wherein the reinforced material is configured to have a maximum stretch of up to 10%. In some embodiments, the pre-stretch of the fibers ranges between 50% and 95% of the maximum stretch of the fibers. In some embodiments, a middle layer comprising pre-stretched fibers is held taught while a top elastomeric layer is applied, and held taught while the elastomer is absorbed by and saturates the fibers, followed by applying the bottom adhesive layer once the elastomer is fully absorbed by and saturating the fibers.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the system and method of the present invention. The following working examples therefore, specifically point out the exemplary embodiments of the present invention, and are not to be construed as limiting in any way the remainder of the disclosure. Disclosed in this example is a reinforced material for sports, medicine and rehabilitation, in order to provide precise, predictable support to a subject while remaining easy to use and adaptable to diverse environments. The disclosed reinforced material (i.e., Aramid reinforced instant flexible cast strips) provide a precise customized fit that is easily and quickly achieved in length and circumference, while securing anatomical conformity for repair preservation.

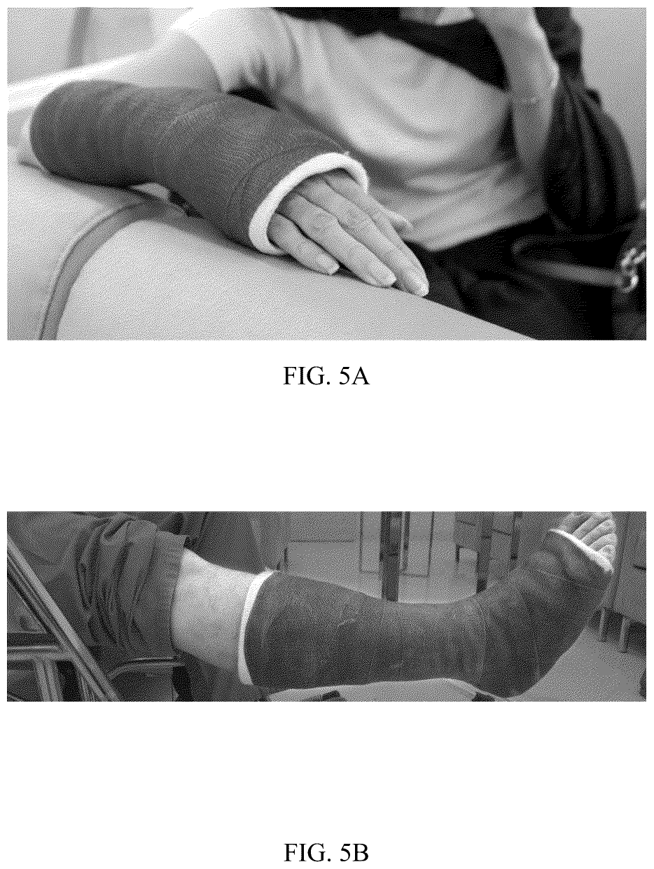

Current alternatives to the disclosed reinforced material include casting as the traditional treatment for injured limbs. FIGS. 5A-5B show the ‘casting’ method which is the traditional treatment for injured limbs. It is typically initiated approximately one week after the fracture, once initial swelling has subsided. Casting utilizes fiberglass material for casting, although plaster may be used in certain cases.

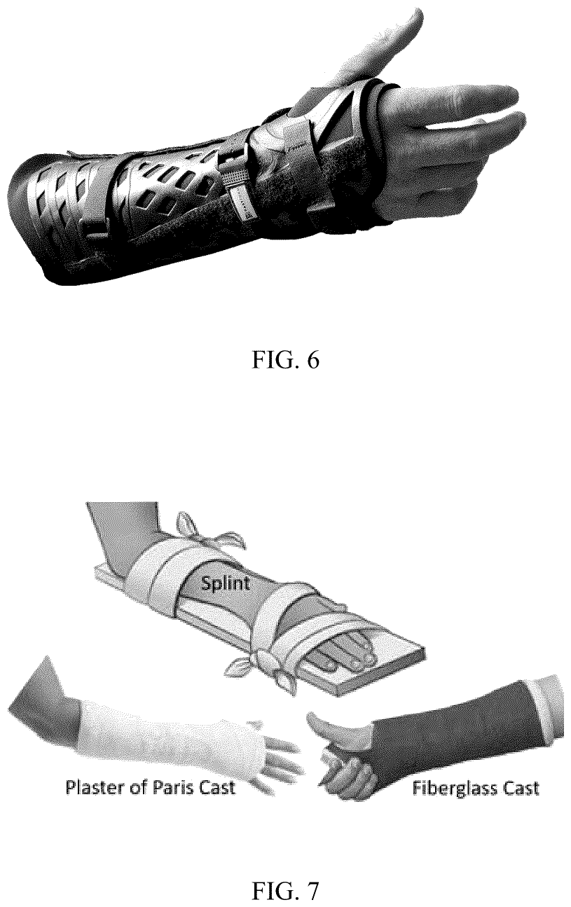

Pre-formed splint cast braces may also be used as an alternative. FIG. 6 shows a pre-formed splint cast brace uses a thermoplastic that when heated converts from a rigid state to a very pliable and conformable material (plaster-like). After sculpting to a desired position and shape, it reverts to a high strength immobilizer. FIG. 7 shows a comparison of the typical immobilizer methods that includes a splint, plaster cast, and fiberglass cast.

In some embodiments, the reinforced material is formed into a strip, not a tape. However, these strips can be in a tape form as compared to the invention being simply a roll a tape. The typical width of these strips can range from . 25″ to 2″ or greater, depending on your application and size of injured area. Generally, the reinforced material comprises, top, middle, and bottom layers, the middle layer comprised of high tensile strength fibers comprising a first and second side; or aramid fibers comprising a first and second side; or a versatile, low-weight, high-strength high modulus polyethylene fiber. It should be appreciated that any Kevlar®, Kevlar-like aramid fibers, or aramid fibers may be used in the disclosed reinforced material. In some embodiments, the middle layer is formed from high tensile strength fiber such as aramid or a versatile, low-weight, high-strength high modulus polyethylene fiber that offers maximum strength combined with minimum weight. On a weight-for-weight basis, it is up to 15 times stronger than steel and up to 40% stronger than aramid fibers.

The reinforced material is impervious to water, moisture and human sweat that can render traditional tapes, casts, and splints ineffective and cause them to breakdown. The reinforced material is lightweight, portable, compact and universal-able to fit most injured areas.

Compared to pre-formed casts that are shaped for a specific body part like a wrist or ankle and therefore are limited with their applications as well as being bulky and heavy. Normally high tensile strength fibers require a special cutting tool. But because the invention suspends these fibers and keeps them taut (stretched or pulled tight; not slack), the strips can be with an ordinary scissor or knife.

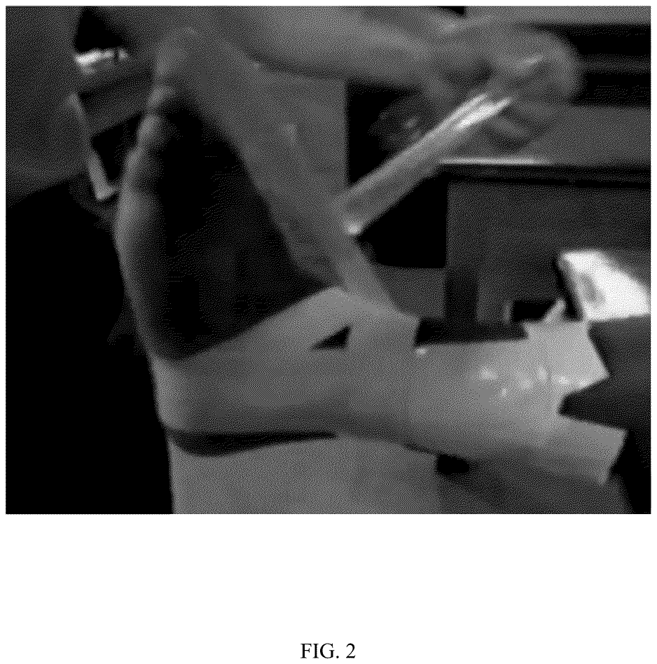

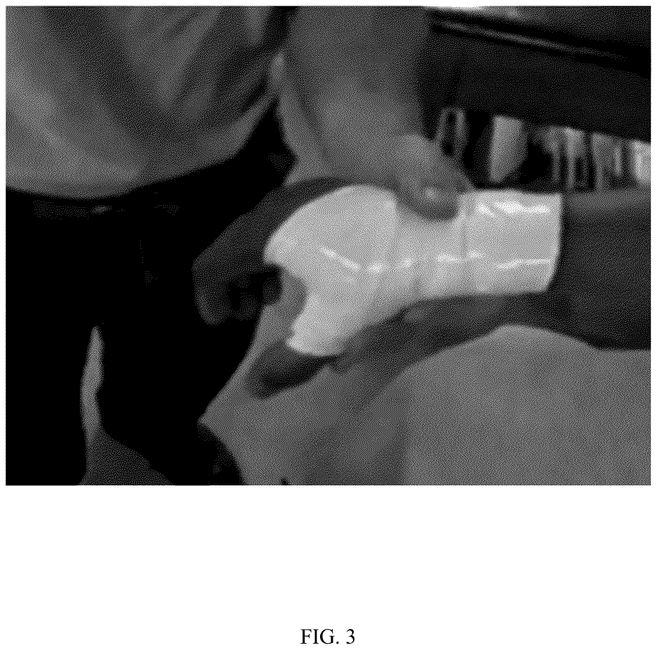

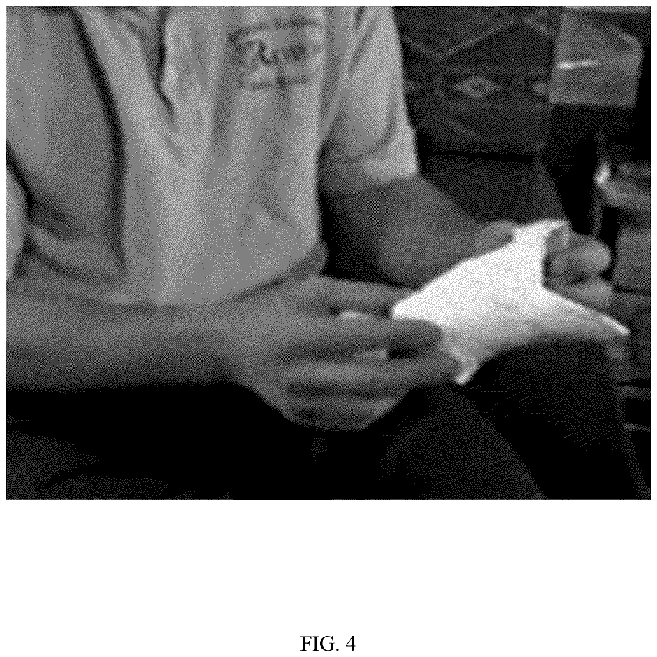

FIG. 2 shows the reinforced material of the present invention formed into Aramid reinforced instant flexible cast strips applied around an ankle to support the ankle without excessively hindering movement. A removable or peelable layer is shown being peeled from the reinforced material. The pre-set maximum elongation of the reinforced material results in less tape being required to support the ankle of the patient or subject. The reinforced material can stick to itself for additional support. FIG. 3 shows the strips allied around a wrist. By layering the reinforced material, rigid cast may be formed around a portion of patient or subject. FIG. 4 shows the cast formed by the multi-layered material retains its structure and form when removed from the subject, and exhibits or retains flexibility and elasticity.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A reinforced material, comprising:
a middle layer formed of a plurality of fibers comprising a first and second side;
a top layer disposed on the first side comprising an elastomeric material; and
a bottom layer disposed on the second side comprising an adhesive material, wherein the reinforced material is configured to have a maximum stretch of up to 10%.

2. The material of claim 1, wherein the plurality of fibers comprise any of aramid fibers, high tensile strength fibers, or a versatile, low-weight, high-strength high modulus polyethylene fibers.

3. The material of claim 1, wherein the plurality of fibers are pre-stretched.

4. The material of claim 3, wherein the pre-stretch of the plurality of fibers ranges between 50% and 99% of the maximum stretch of the fibers.

5. (canceled)

6. The material of claim 1, further comprising a removable or peelable layer disposed on the top or bottom layer.

7. (canceled)

8. The material of claim 1, wherein the plurality of fibers are arranged in a unidirectional configuration, a multi-directional configuration, or one or more woven patterns to maximize tensile strength along one or more axis.

9. The material of claim 8, wherein the one or more woven patterns comprises warp and weft fibers, each having a thread count or number of fibers per unit area, wherein the thread count or number of fibers per unit area of the weft fiber is less than the thread count or number of fibers per unit area of the warp fibers.

10. The material of claim 1, further comprising elastic fibers woven into the middle layer.

11. The material of claim 1, wherein the reinforced material is configured to be hypoallergenic and suitable for prolonged contact with human skin.

12. The material of claim 1, wherein the elastomeric coating is configured to provide a textured or high-friction surface to improve grip and handling during application.

13. The material of claim 1, wherein the reinforced material is fabricated in the form of a roll, a roll of strips, a patch, or a strip for specific applications.

14. The material of claim 13, wherein the strip is at least partially formed in an elongate shape, a winged-shape, an hourglass shape, or an irregular shape.

15. The material of claim 1, wherein the top layer is treated or configured to be water-resistant or moisture-wicking.

16. The material of claim 1, wherein the reinforced material is perforated to allow for ventilation during use, or has one or more cutout regions configured as reliefs.

17. The material of claim 1, wherein the adhesive material comprises an antimicrobial agent.

18. The material of claim 1, wherein the elastomeric coating is lettered, colored or patterned to indicate specific application zones, instructions, cutting zones, or to provide aesthetic customization.

19. (canceled)

20. The material of claim 1, wherein the reinforced material is configured to be waterproof, moisture-proof, and sweat-proof.

21. A roll of tape formed from pre-cut strips of reinforced material, the reinforced material comprising:
a middle layer formed of a plurality of aramid fibers comprising a first and second side;
a top layer disposed on the first side comprising an elastomeric coating; and
a bottom layer disposed on the second side comprising an adhesive coating.

22. (canceled)

23. A method of fabricating a reinforced material, comprising:
pre-stretching a plurality of high-tensile strength fibers into a middle layer having a first side and a second side;
applying an elastomeric layer, material or coating to the first side; and
applying an adhesive layer, material or coating to the second side, wherein the reinforced material is configured to have a maximum stretch of up to 10%.

24. The method of claim 23, wherein the pre-stretch of the fibers ranges between 50% and 95% of the maximum stretch of the fibers.
